(19) United States
(12) Patent Application Publication
(10) Pub. No.: US 2009/0275953 A1
(43) Pub. Date: Nov. 5, 2009

(54) SPOOL INTERVERTEBRAL DISTRACTION DEVICE AND METHOD

(76) Inventors: JAMES MARINO, LA JOLLA, CA (US); JAMIL ELBANNA, SAN DIEGO, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/397,130

(22) Filed: Mar. 3, 2009

Related U.S. Application Data
(60) Provisional application No. 61/068,048, filed on Mar. 3, 2008.

(51) Int. Cl. A61B 17/58; A61F 2/44; A61B 19/02

(52) U.S. Cl. 606/90; 623/17.11; 206/438

(57) ABSTRACT

An intervertebral disc space distraction system includes a spool core sized to be percutaneously positioned within a portion of a disc space between adjacent vertebral bodies, the spool core having a first diameter. The system further includes an elongate film removably attached to the spool core, wherein the elongate film can be wound around the spool core to increase the first diameter of the spool core.

### REFERENCE TO PRIORITY DOCUMENT

This application claims priority of U.S. Provisional Patent Application Ser. No. 61/068,048 entitled “Spool Intervertebral Distraction Device and Method” by J. Marino, filed Mar. 3, 2008. Priority of the filing date of Mar. 3, 2008 is hereby claimed, and the disclosure of the Provisional Patent Application is hereby incorporated by reference.

### BACKGROUND

The present disclosure relates to methods and devices for separating an intervertebral disc space and for maintaining the space during postoperative healing.

Pursuant to treatment of the spine, it is sometimes necessary to distract a first vertebral body relative to a second vertebral body in order to gain access to the disc space between the vertebral bodies. There is a growing need to perform such distraction through small access portals of sufficient dimension in order to minimize trauma to the patient. In view of the foregoing, there is a need for vertebral distraction devices and methods that are adapted to be performed through small access portals.

### SUMMARY

The present disclosure relates to methods, systems and devices for separating an intervertebral disc space and for maintaining the space during postoperative healing.

Disclosed is an intervertebral disc space distraction system that includes a spool core sized to be percutaneously positioned within a portion of a disc space between adjacent vertebral bodies, the spool core having a first diameter. The system further includes an elongate film removably attached to the spool core, wherein the elongate film can be wound around the spool core to increase the first diameter of the spool core.

Also disclosed is a method for distracting adjacent vertebral bodies. The method includes introducing percutaneously a rotatable spool core into a portion of a disc space between the adjacent vertebral bodies and introducing percutaneously an elongate film into the portion of the disc space between the adjacent vertebral bodies. The method also includes attaching the elongate film to the spool core to form a spooled assembly having a first diameter, rotating the spool core to wind the film around the spool core, and increasing the first diameter of the spooled assembly to a second diameter by adding successive layers of the elongate film to the spool core, wherein the second diameter is greater than the first diameter. The method also includes exerting a force by the spooled assembly against at least one of the adjacent vertebral bodies causing relative distraction of the adjacent vertebral bodies.

Other features and advantages of the present invention should be apparent from the following description of various embodiments, which illustrate, by way of example, the principles of the invention.

### BRIEF DESCRIPTION OF THE DRAWINGS

FIG. 1 shows a perspective view of a spool distraction system.

FIG. 2 shows a perspective view of the spool with a distal end of the film positioned adjacent the spool.

FIG. 3 shows the film attached to the spool.

FIG. 4 shows the spool with a substantial length of film wound around the spool.

FIG. 5 shows the locking element positioned adjacent the portal prior to insertion therein.

FIG. 6 shows the locking element fully locked onto the film and to the spool.

FIG. 7 shows a perspective view of the spool delivery member.

FIG. 8A shows an enlarged view of the attachment end of the spool delivery member.

FIG. 8B shows a perspective view of the film delivery member.

FIG. 9 shows an oblique iso-view of a spine showing a pair of intraosseous transpedicular pathways.

FIGS. 10A-10D show various views of the spooling process.

FIGS. 11A-11D show additional views of the spooling process.

FIGS. 12A-12D show additional views of the spooling process.

FIGS. 13A-13D show various views of the completed spooling process.

FIGS. 14A-14D show various views of the dissociation of the spool from the insertion instruments.

FIGS. 15A-15D show various views of the spool being positioned in the disc space.

### DETAILED DESCRIPTION

Disclosed is a vertebral distraction system that is adapted to distract vertebral bodies such as to improve the relative position of one vertebra relative to another vertebra in terms of sagittal balance, increase spinal canal and neuroforaminal cross-sectional areas, and improve access to the disc space between the vertebral bodies. The disclosed system is adapted to gain access to the intervertebral disc space via small access portals. The system can distract or separate the intervertebral disc space and maintain the distraction during a period of postoperative healing.

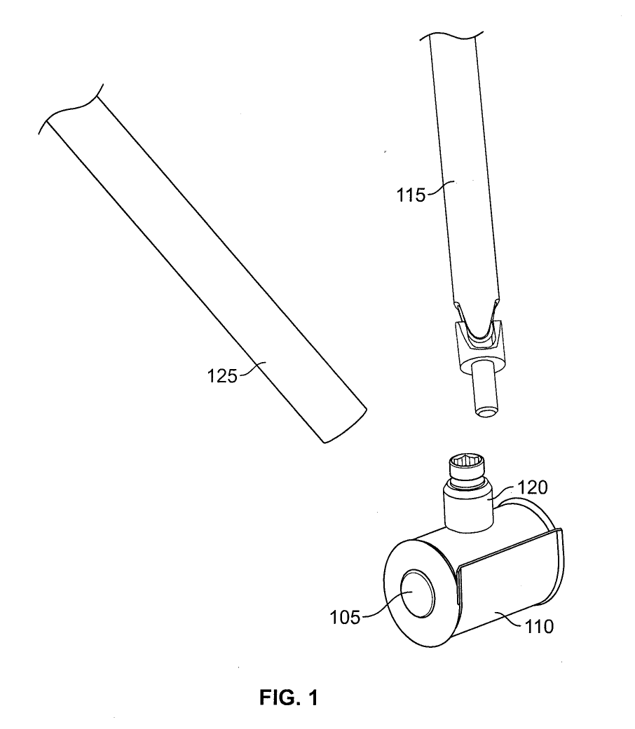

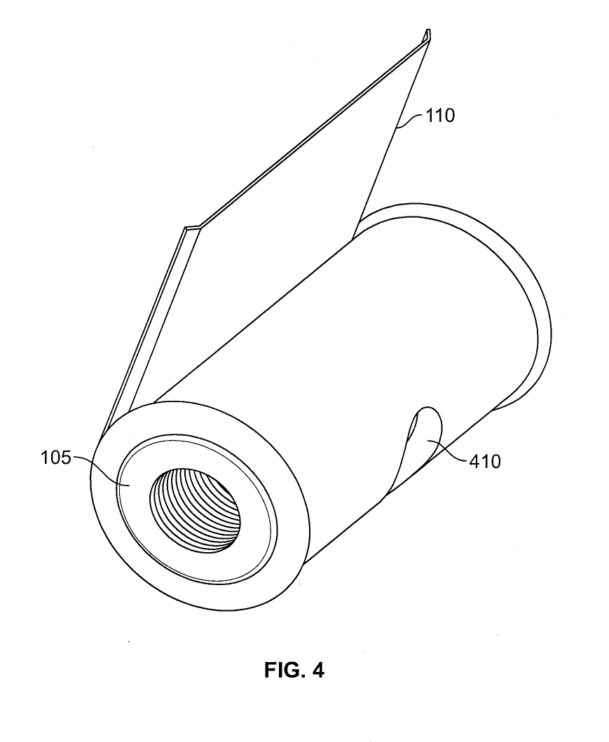

FIG. 1 shows a perspective view of a spool distraction system. The system includes a spool core or spool 105, an elongate film 110, and a spool delivery member 115. The film 110 is adapted to removably attach to the spool 105 at a distal end of the film 110. The spool delivery member 115 removably attaches to the spool 105 and can be used to delivery and rotate the spool 105 to cause the attached film 110 to wind around the outer surface of the spool 105. In an embodiment, the spool delivery member 115 attaches to the spool 105 by inserting through a bore running through at least a portion of the longitudinal axis of the spool 105. The bore can have threads (such as shown in FIG. 4) or other engagement features on its surface such that it improves the coupling engagement between the spool delivery member 115 and the spool 105. The system further includes a locking element 120 that mechanically locks the film 110 to the spool 105 when a desired amount of film 110 has been wound around the spool 105, as described in detail below. The system can further include a film delivery member 125 that is adapted to deliver the film 110 into a position within or adjacent the disc space of a patient, as described more fully below. For clarity of illustration, FIG. 1 shows only distal regions of the spool delivery member 115 and the film delivery member 125, which are shown and described in more detail below.

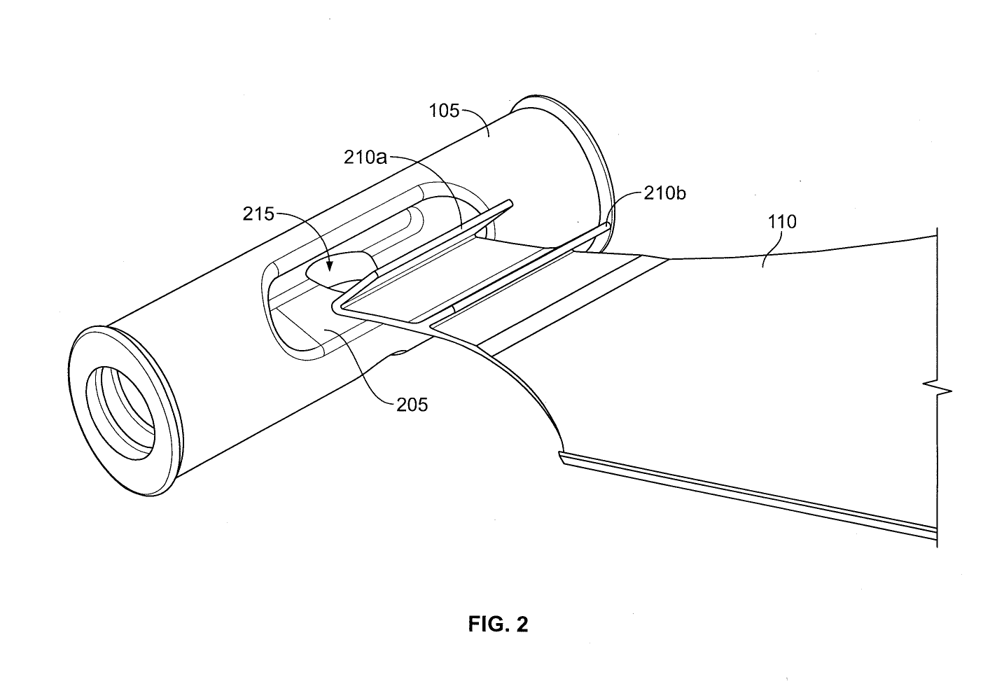

FIG. 2 shows a perspective view of the spool 105 with a distal end of the film 110 positioned adjacent the spool 105 such that the film 110 is ready to be coupled to the spool 105. In this regard, the spool 105 has a coupling element that is adapted to removably attach to the distal end of the film 110. In the illustrated embodiment, the coupling element is a slot-shaped hole 205 that extends through the spool 105 and that is sized and shaped to receive the distal end of the film 110. The hole 205 extends entirely through the spool 105 such that two openings are formed in the outer surface of the spool 105 with the hole 205 providing a pathway therebetween.

The film 110 is attached to the spool 105 by inserting the distal end of the film 110 through the opening such that the film 110 engages the spool 105 and removably secures to the spool 105. The film 110 is typically attached to the spool 105 after delivery of the spool 105 into the disc space. The film 110 and/or the spool 105 can be equipped with one or more engagement structures that facilitate a secure attachment between the film 110 and the spool 105. For example, the illustrated embodiment of the film 110 includes a pair of backwardly-extending, barb-like engagement members 210a and 210b (collectively engagement members 210). The distal-most engagement member 210a is sized and shaped to engage the spool 105 when the film 110 is properly inserted into the hole 205. The proximal-most engagement member 210b is sized and shaped to be grasped by an insertor's grasping instrument during insertion into the hole 205 and for removal of the film 110 from the spool 105. In addition, the region of the film 110 that attaches to the spool 105 can have a reinforced shape or structure that facilitates a secure anchor between the film 110 and the spool 105. For example, the film 110 can have a thickness near the anchor point that is greater than the rest of the length of the film. The film 110 can have a tapered distal end or a reinforcement member attached to or near the distal end of the film 110.

The film can be made of various materials. The film can be of a material that is flexible and that can be folded, rolled, bent or otherwise reshaped to reduce the dimensional requirements of an insertion channel or cannula through which it would be introduced. For example, the film 110 can be a polymer such as an elastomeric polymer. The film 110 can be a metal ribbon such as nitinol or stainless steel or other elastic metal. The flexibility of the material permits the film to be rolled, folded or otherwise manipulated so that it is of a first reduced size, such as when being passed through the film delivery member but will return to its initial shape or size once unrolled, unbent, unfolded etc. For example, the film can be rolled or bent around its long axis so that it achieved a reduced overall width for passing through the film delivery member. Once the film, or a portion thereof, exits the film delivery member at its distal end, the film can unfold, unroll or otherwise expand back to its original width and size. In an embodiment, the film 110 has a first width in an unfolded or unrolled state and the film delivery device has a second width that is smaller than the first width of the film 110. The film can be in a folded or rolled state and reduced to a third of its overall width that is smaller than the diameter of the film delivery device. It should be appreciated that although the device and method is described herein as comprising a film, the film need not be limited to a flat, ribbon-shaped structure. The film can alternatively be a cord, braid, tether or other elongate structure capable of winding onto the spool core to increase the initial diameter for the spool core to a second expanded diameter.

The film 110 can have various material properties adapted to promote desired features. For example, the surface texture of the film 110 can be smooth, which may allow for uniform tensioning or it could have a matte finish, to increase friction. The film 110 can have complimentary small depressions and prominences that could interdigitate when properly aligned. In an embodiment, the film 110 has a very fine transverse tread feature, such as a very fine transverse spline on the upper and lower surface of the film representing a percentage (such as 25%) of the film's overall thickness. Such a feature would stabilize the film by promoting the film locking layer to layer during winding around the spool 105. In another embodiment, the film 110 can have a visual indicator on an outside surface that indicates the relative length of film 110 wound onto the spool.

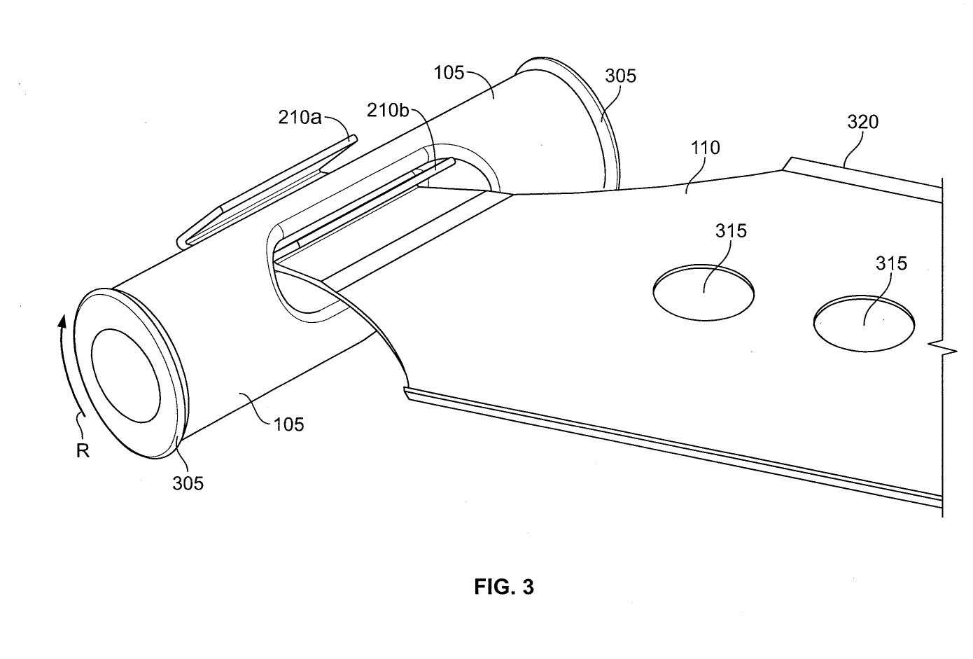

FIG. 3 shows the film 110 with its distal end attached to the spool 105. Note that the distal engagement member 210a is positioned such that it engages a portion of the spool 105 to provide a secure attachment between the film 110 and the spool 105. The distal engagement member 210a of the film 110 extends through the hole 205 from one end and engages the spool 105 from the opposite end of the hole 205. It should be appreciated that other mechanisms can be used to removably attach the film 110 to the spool 105 and that the spool distraction system is not limited to the particular mechanism shown in FIGS. 2 and 3.

In an embodiment, the spool 105 can have a pre-attached tether or tether elements that are associated with the spool. The pre-attached tether(s) could be wound around or spooled on the spool core. One end of the tether or tethers could be retrieved from disc space with the aide of a grasping instrument and pulled from within the disc space through the film delivery cannula such that the tether(s) are then available externally. The tether or tethers could then be associated with the film 110 such that winding of the spool 105 retrieves the tether(s) to within the disc space and the film 110 associated with the tether(s).

With reference still to FIG. 3, when the spool 105 is rotated (as represented by the arrow R in FIG. 3), the film 110 winds around the outer surface of the spool 105 by virtue of the attachment between the distal end of the film 110 and the spool 105. A raised bevel or flange 305 can be positioned at each of the opposed lateral edges of the spool. In an embodiment, the flange 305 has a height (from the outer surface of the spool) of approximately twice the thickness of the film 110 although the height can vary. The flanges 305 help to align the initial winding of the film 110 around the spool 105. The flanges 305 present an inclined plane to the edges of the film during the winding process. The flanges 305 produce slight medial lateral corrections as the tensioned film 110 winds around the spool 105.

The film itself can also have self-aligning features that facilitate uniform or even winding of the film 110 around the spool 105. For example, the film 110 can have raised bevels 320 on opposite lateral edges that are adapted to mate with or otherwise align with the flanges 305 of the spool. In an embodiment, the film bevels 320 extend vertically about twice the thickness of the film 110 and are oriented at an angle of about 60 degrees relative to the extended plane of the film. This would help self-align the film with additional spooling beyond the influence of the spool 105. Each successive layer of film 110 would “nest” within the trough or gutter created by the bevel 320.

With reference still to FIG. 3, a series of holes 315 can be positioned in series along the length or a portion of the length of the film 110. The holes 315 are spaced from one another in a manner such that the holes 315 align with one another as the film 110 winds around the spool 105. FIG. 4 shows the spool 105 with a substantial length of film 110 wound around the spool 105. With the film 110 wound around the spool 105, the holes 315 (FIG. 3) are all aligned with one another to collectively form a portal 410 that extends through the film 110. The portal 410 provides an opening where the locking element 120 (FIG. 1) can be inserted to lock the film 110 in the wound state to the spool 105.

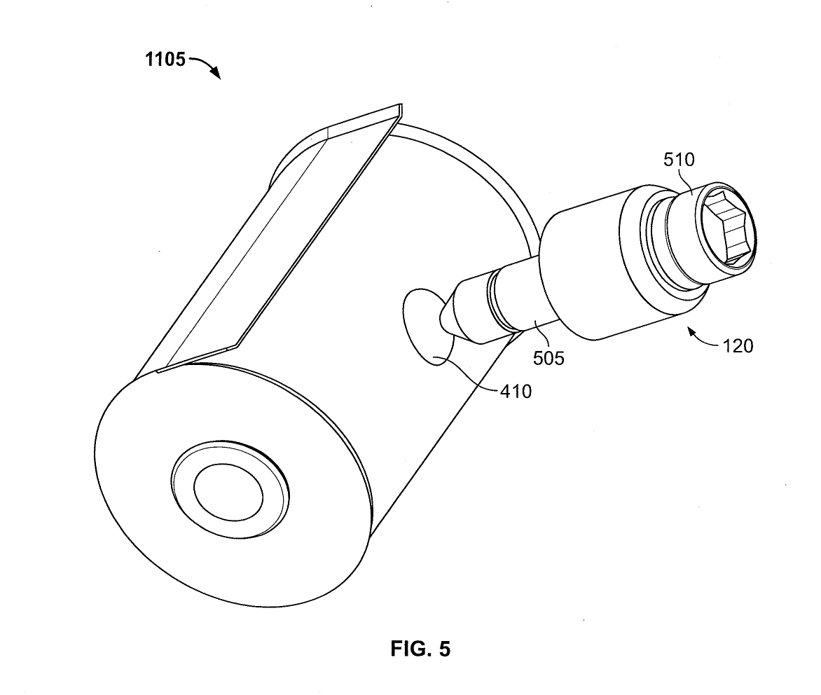

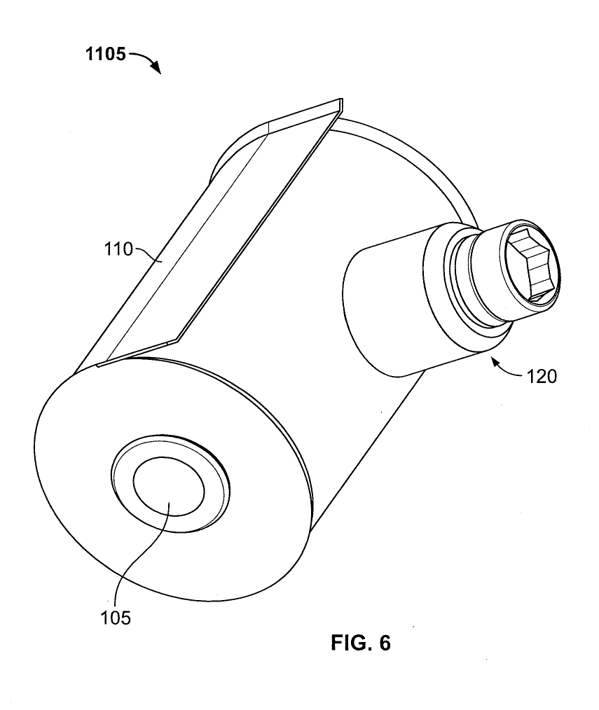

FIG. 5 shows the locking element 120 positioned adjacent the portal 410 prior to insertion therein. The locking element 120 has an engagement region such as an elongate pin 505 that is sized and shaped to fit into the portal 410 and lock into the spool 105. A distal end of the pin 505 can be chamfered or otherwise shaped to facilitate insertion into the portal. The distal end of the pin 505 is adapted to fit into and lock with a corresponding hole 215 positioned inside the spool 105 and aligned with the portal 410 (see FIG. 2). The pin 505 can have locking means, such as threads or other means, that mate with and lock with the spool and film to secure the locking element 120 to the spool-film assembly 1105. In addition, a proximal end of the locking element 120 can have a coupling element 510 that couples to a tool that can be used to rotate or otherwise drive the locking element 120 into the portal 410. FIG. 6 shows the locking element 120 fully locked onto the film 110 and to the spool 105 forming the spool-film assembly 1105.

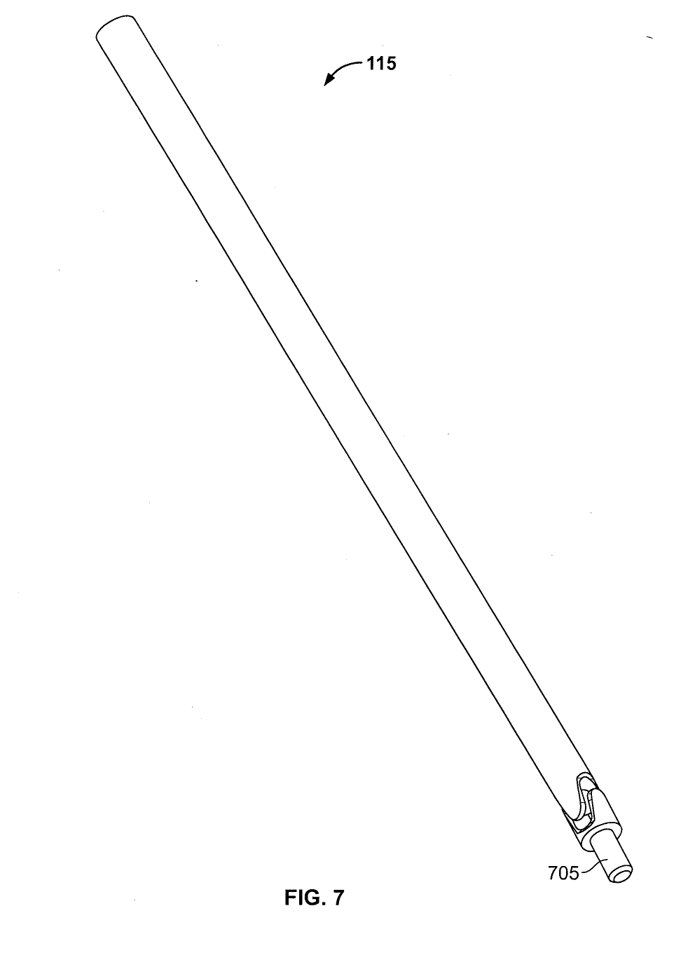

As mentioned, the spool distraction system includes a delivery member 115 that removably attaches to the spool 105. FIG. 7 shows a perspective view of the spool delivery member 115. Once it is attached to the spool 105, the delivery member 115 can be rotated to impart rotation to the spool 105 and thereby cause the attached film 110 to wind around the outer surface of the spool 105. In an embodiment, the spool delivery member 115 is an elongate rod having a distal attachment end 705 that removably couples to the spool 105. The attachment mechanism between the delivery member 115 and the spool 105 can vary.

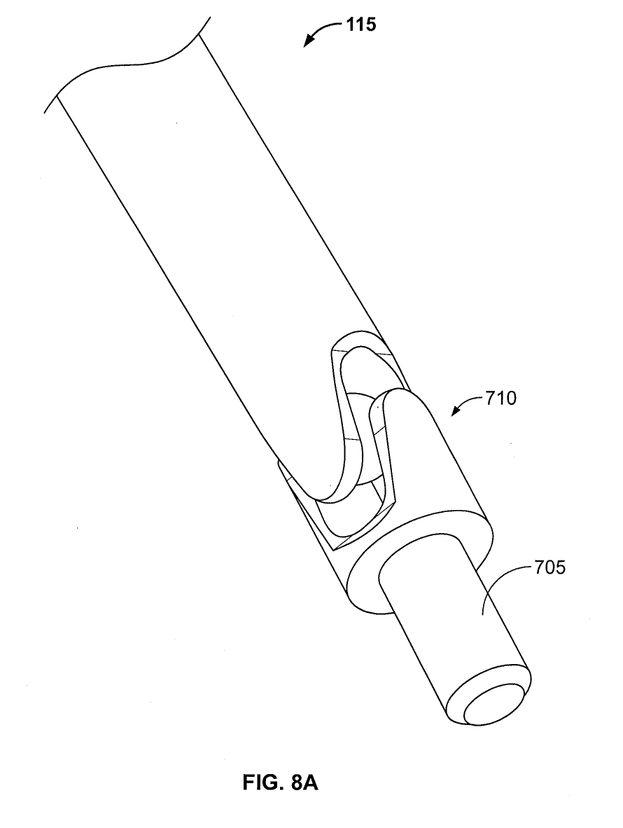

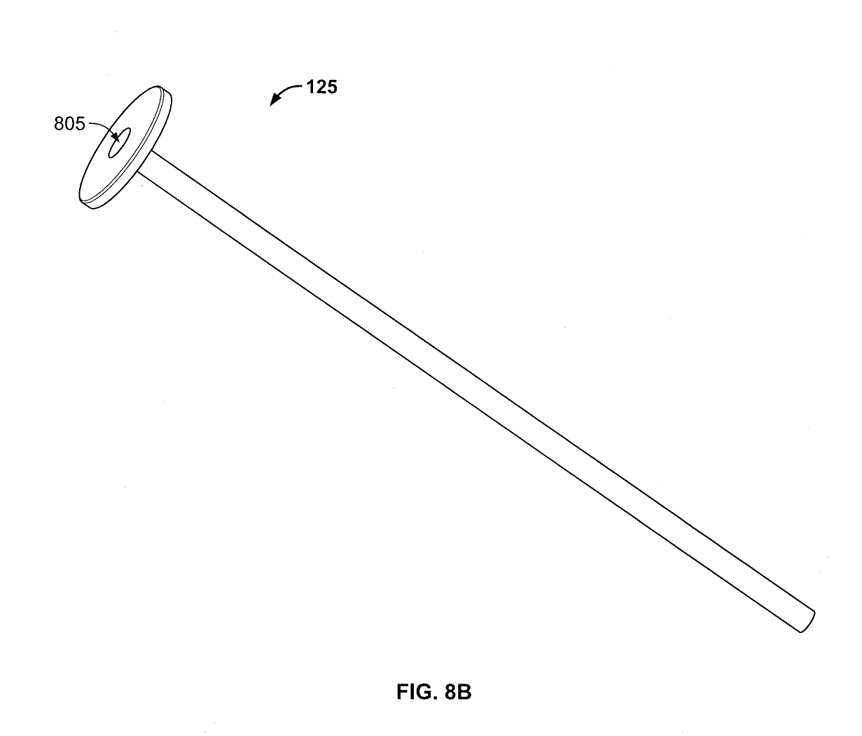

FIG. 8A shows an enlarged view of the attachment end 705 of the spool delivery member 115. In an embodiment, the attachment end 705 has a universal joint or flexible shaft component 710 is used to attach the delivery member 115 to the spool 105. This allows the spool 105 to be oriented parallel to the long axis of the spool delivery member 115 during delivery of the spool 105 into the patient. Once positioned at a desired location in the spine, the flexible shaft component 710 can then be re-oriented, such that the spool 105 is perpendicular to the direction from which the film is introduced. This is described in more detail below with reference to the method of use of the spool distraction system.

FIG. 8B shows a perspective view of the film delivery member 125, which comprises an elongate element with an internal passageway 805. The internal passageway 805 is sized to receive the film 110 therethrough. The film delivery member 125 is sufficiently long to provide percutaneous access to a spinal disc space. The outer diameter or transverse dimension of the film delivery member 125 is sized to fit within a pathway (e.g. a transpedicular pathway) to the disc space. The inner diameter of the film delivery member 125 can vary but is generally sized to receive the film 110. The width of the film 110 also can vary and can depend on whether it is in an unfolded or folded, unbent or bent, and unrolled or rolled configuration.

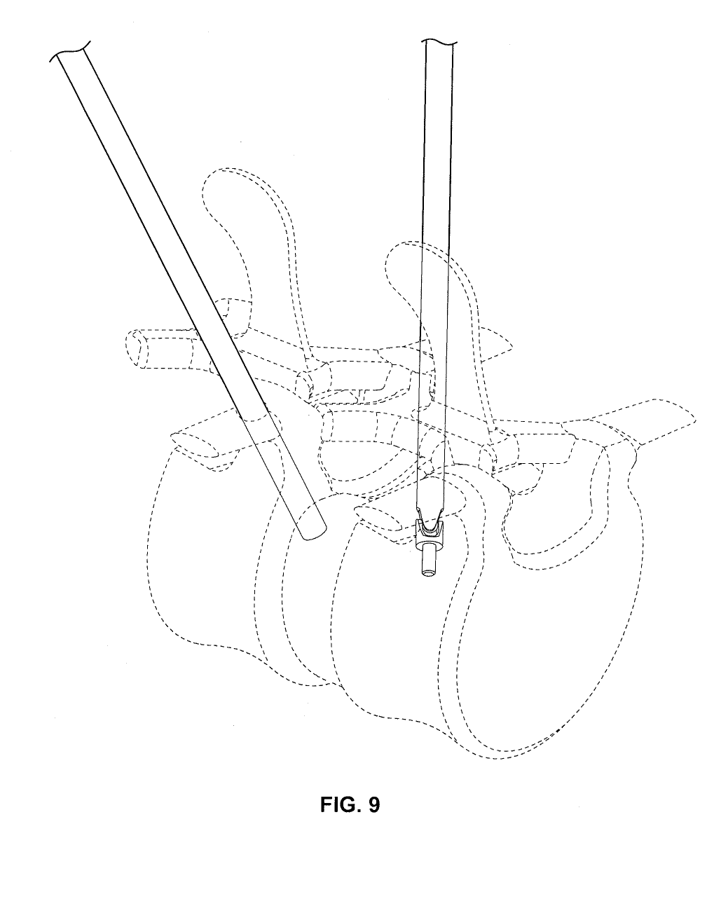

A method of using the spool distraction system is now described. At least one pathway is formed in the patient to provide access to the disc space to be treated. Various methods and devices can be used to form the at least one pathway. The disc space can be a prepared disc space such as partially vacated disc space. In an embodiment, a pair of intraosseous transpedicular pathways is formed wherein each pathway provides a portal into the disc space. FIG. 9 shows an oblique iso-view of a spine showing the film and spool delivery devices 115, 125 positioned in the intraosseous transpedicular pathways that provide access to the disc space. In an embodiment, access pathways P1 and P2 are formed on either side of the disc's mid-sagittal plane. The pathways can be formed pursuant to the methods and devices described in U.S. Patent Publication No. 2007-0162044, which is incorporated herein by reference in its entirety. It should be appreciated that for clarity of illustration the access pathways P1 and P2 are occasionally omitted from the figures. Further, the figures illustrate the anatomic landmarks in the spine and access through the vertebrae to the intervertebral disc space in schematic. Those skilled in the art will appreciate that actual anatomy include anatomical details not shown in the figures.

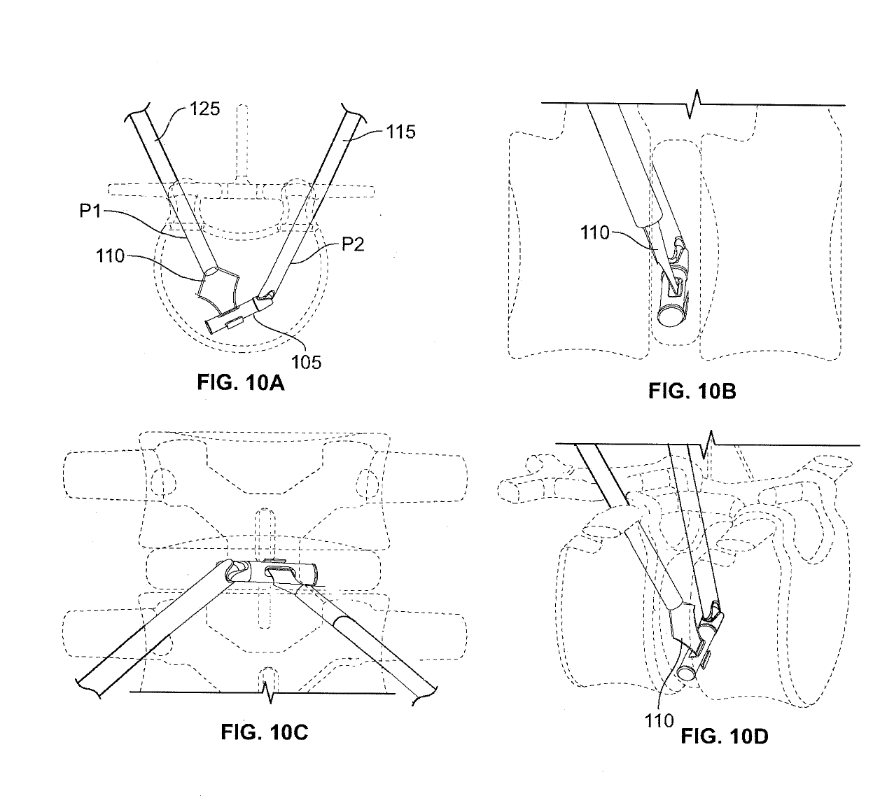

With reference to FIGS. 10A-10D, once the pathways are formed, the spool delivery member 115 and the film delivery member 125 are each inserted into a respective pathway to provide access to the disc space. That is, the film delivery member 125 is inserted into the pathway P1 such that a proximal end is external to the patient and the distal end is at or near the disc space. Likewise, the spool delivery member 115 (with the spool 105 attached to the distal end) is inserted into the pathway P2 such that a proximal end is external to the patient and the distal end is at or near the disc space. The internal passageway 310 of the film delivery member 125 has an opening at a proximal end and an opening at a distal end providing a conduit for the film 110 to be delivered into the disc space.

FIGS. 10A-10D shows various views of the disc space after the film 110 has been passed through the film delivery member 125 into the disc space. The internal passageway 310 of the film delivery member 125 is sufficiently large to provide for transit of the film 110. Note that the film 110 is in an unwound state such that the distal end of the film 110 protrudes out of the film delivery member 125 into the disc space. The spool delivery member 115 has been passed through the pathway P2 such that the distal end of the spool delivery member 115 is at or near the disc space with the spool 105 attached to the distal end. As mentioned, the attachment end of the spool delivery member has a universal joint or flexible shaft component 710 that allows the spool 105 to be oriented parallel to the long axis of the spool delivery member 115 during delivery of the spool 105 through the pathway P2. Once positioned within the disc space, the flexible shaft component 710 permits the spool 105 to be re-oriented into a position that allows the distal end of the film 110 to be coupled to the spool 105. The film 110 is advanced in a distal direction until the film 110 is coupled to the spool 105. FIGS. 10A-10D show the spool 105 in the disc space such that the distal end of the film 110 has been coupled with the spool 105. As discussed above, the distal end of the film 110 couples to the spool 105 in a manner (either directly or via tether(s)) that provides a secure attachment therebetween.

In FIG. 10, the film 110 is shown coupled to the spool 105 and the spool 105 is still attached to the spool delivery member 115. The delivery member 115 can then be used as a drive tool or have a drive shaft to rotate the spool 105 about its axis. The rotation of the spool 105 causes the film 110 to wind around the spool 105. This causes successive layers of film 110 to form around the spool 105 with the successive layers of film 110 causing a gradual increase in the overall diameter of the spool-film assembly 1105. The increasing diameter of the spool-film assembly 1105 causes distraction of the disc space due to the outer layer of film exerting an outward force against the vertebral bodies adjacent to the disc space. The delivery member 115 can also include a device to measure the torque associated with spooling of the film onto the spool core using the drive shaft such that the desired extent of interbody distraction is achieved.

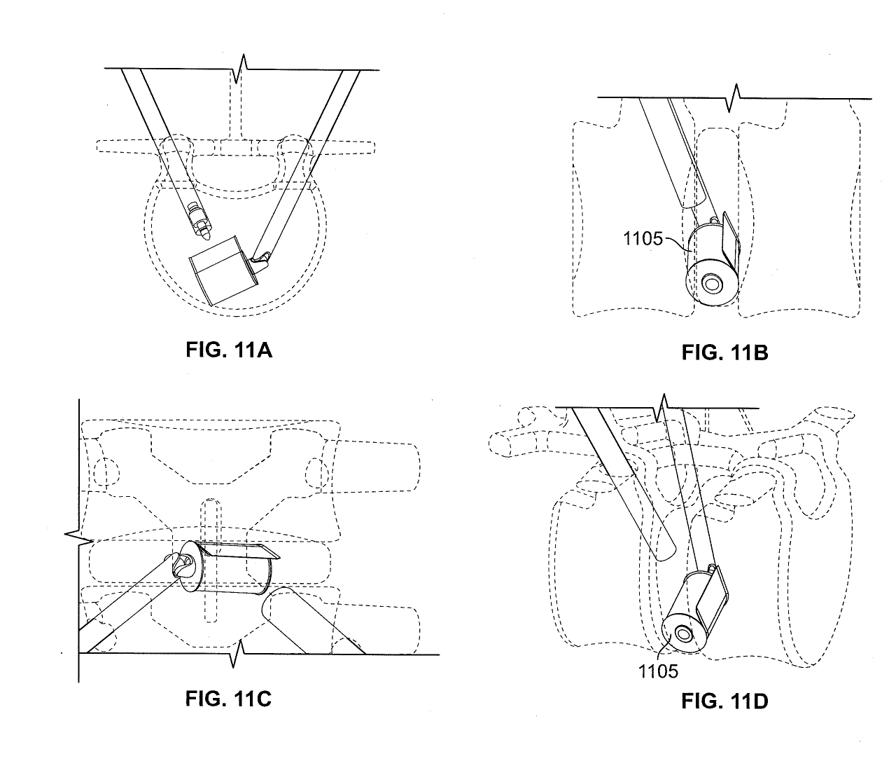

FIGS. 11A-11D show various views of the spool-film assembly 1105 after a substantial length of film 110 has been wound around the spool 105. Visible indicators can be present on a surface of the film 110 to identify the length of film 110 wound around the spool 105. These indicators in turn determine the overall increase in diameter of the spool-film assembly 1105 obtained during winding of the spool 105. The visible indicators can include any variety of appropriate indicators including hatching, numbering or other metered marks visible to the surgeon during delivery. The increased diameter of the spool-film assembly 1105 has caused distraction of the disc space.

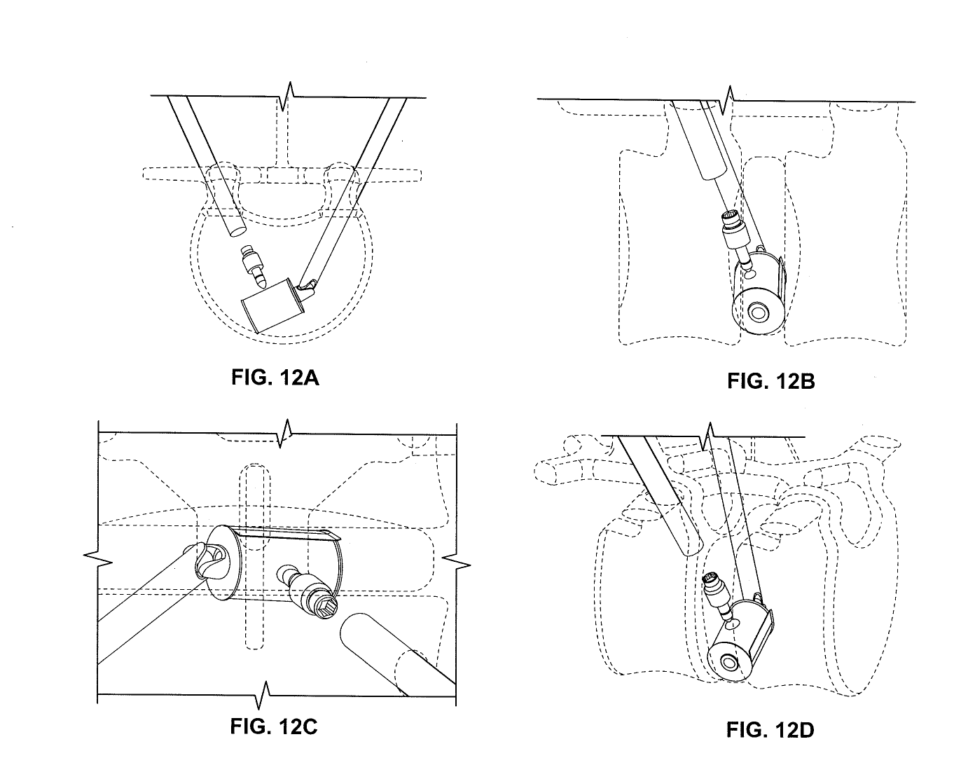

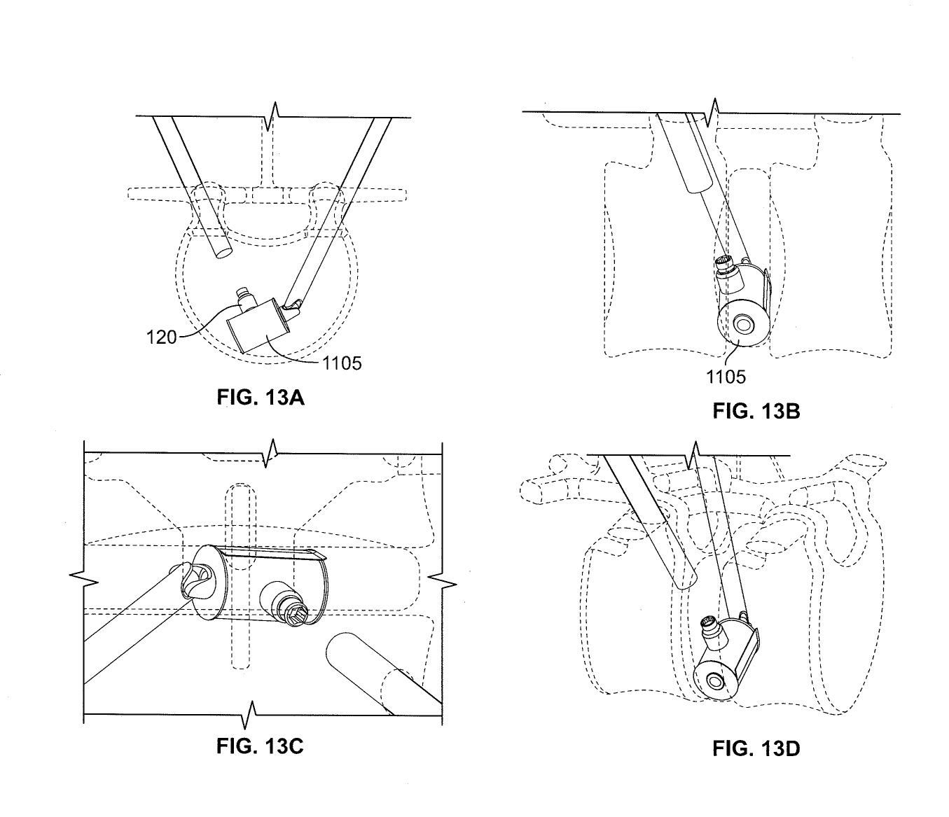

Once the spool-film assembly 1105 has achieved the desired diameter, the remaining film 110 is detached from the spool-film assembly 1105 such as by cutting the film 110. As mentioned, the locking element 120 is used to mechanically lock the film 110 to the spool 105 when a desired amount of film 110 has been wound around the spool 105. With reference to FIG. 12, this is accomplished by delivering the locking element 120 into the disc space via the film delivery member 125, which provides a pathway to the spool-film assembly 1105. The portal 410 (FIGS. 4, 5) that extends through the film 110 and into the spool is aligned with the pathway formed by the film delivery member 125. The locking element 120 is then delivered through the film delivery member 125 and into the portal 410 such that the locking element 120 locks into the spool-film assembly 1105 and secures the film 110 to the spool 105. FIGS. 13A-13D show the spool-film assembly 1105 with the locking element 120 in place within the spool-film assembly 1105.

Alternatively, the distal end of the locking element 120 can have a drill tip or cutting flutes that provide for a self-drilling and self-tapping function, such that rotational and linear advancement of the locking element 120 can create a channel through the spooled film 110 and the spool core 105 with which threaded elements of the locking element 120 engage.

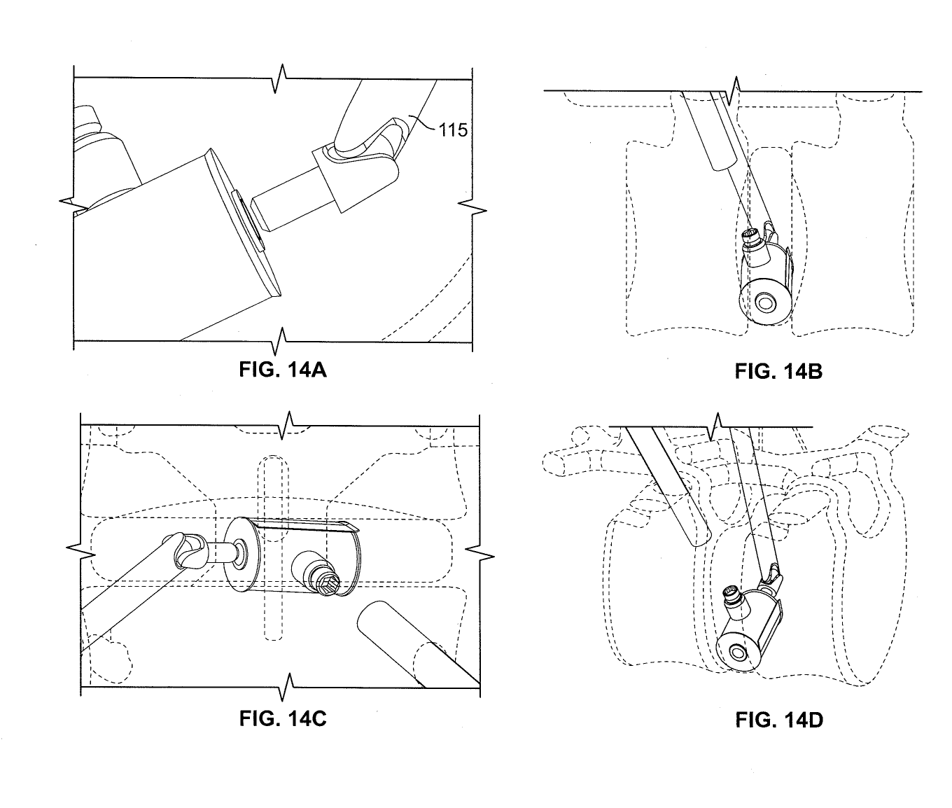

In a subsequent step, the spool delivery member 115 is disconnected from the spool 105. The disconnection of the spool delivery member 115 from the spool 105 can occur using various mechanisms or movements (e.g. reverse rotation of the spool insertion or delivery instrument 115). FIGS. 14A-14D show the spool delivery member 115 being disconnected from the spool 105 while the spool 105 remains within the disc space. The spool delivery member 115 is then retracted or withdrawn from the patient.

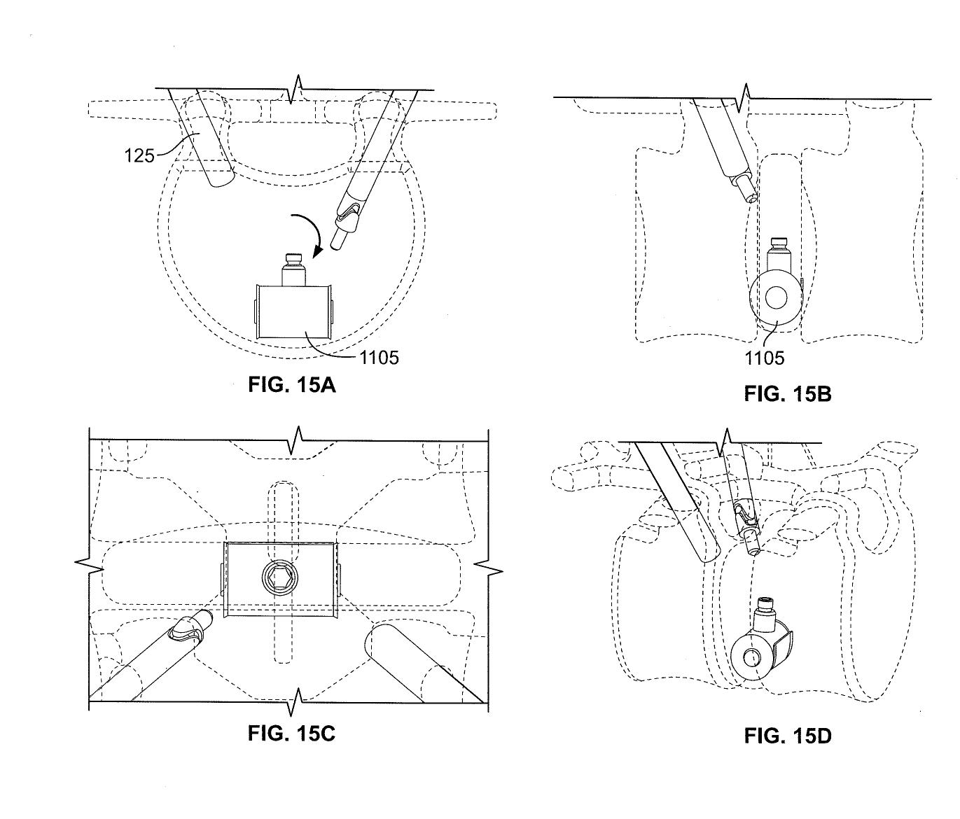

In a next step, the spool-film assembly 1105 is repositioned to a desired orientation within the disc space. FIGS. 15A-15D show a representation of the spool-film assembly 1105 being moved to an exemplary position in the disc space. In the illustrated example, the spool-film assembly is rotated and/or translated to a position that intersects the vertebral midline. It should be appreciated that the spool-film assembly can be positioned in any desired location or orientation in the disc space. A grasper/manipulator device can be used to push and pull the spooled film assembly into a desired position within the disc space.

After the spool-film assembly 1105 is properly positioned, additional material can be introduced into the disc space via the passageway in the film delivery member 125. For example, osteoconductive, proliferative, and/or inductive material can be introduced into the remainder of the disc space for fusion. In another embodiment, a relatively elastic (e.g. polyurethane) material is spooled into the center of a partially evacuated intervertebral disc as a nuclear replacement. The material spooled can be varied to project a surface mesh of material that is fibrinogenic (e.g. woven polyester) to secure or anchor the implant (the spool-film assembly) over time and reduce the risk of migration or expulsion. Such an elastomeric device can be used to replace normal or natural intervertebral disc function, including energy absorption and/or dissipation.

Components of the disclosed intervertebral distraction system can be packaged into a kit. In an embodiment, a prepackaged sterile kit can include the film 110. The film 110 in the kit can be in a wound sterile state such that it can be used to contain the film 110 as it is being delivered and wound onto the spool 105 within the disk space. As described above, visual indicators can be present on an outside surface of the film 110. In an embodiment, the visual indicators can be seen through the packaging or the packaging can have visible indicators showing the length of film 110 being delivered into the film delivery tool and wound onto the spool 105.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

What is claimed is:

1. An intervertebral disc space distraction system, comprising:
a spool core sized to be percutaneously positioned within a portion of a disc space between adjacent vertebral bodies, the spool core having a first diameter; and
an elongate film removably attached to the spool core, wherein the elongate film can be wound around the spool core to increase the first diameter of the spool core.

2. A system as in claim 1, further comprising a spool core delivery member comprising a distal attachment end that removably attaches to the spool core.

3. A system as in claim 2, wherein the spool core delivery member is adapted to percutaneously deliver the spool core into the disc space between adjacent vertebral bodies.

4. A system as in claim 2, wherein the distal attachment end of the spool core delivery member comprises a flexible component, wherein the flexible component permits the spool core to be moved between different orientations relative to a long axis of the spool core delivery member and permits rotation of the spool core by the spool core delivery member.

5. A system as in claim 1, further comprising a film delivery member comprising an internal passageway sized to receive and percutaneously deliver the elongate film to the disc space.

6. A system as in claim 5, wherein the elongate film has a first width and the internal passageway has a second diameter, wherein the first width is greater than the second diameter.

7. A system as in claim 6, wherein the elongate film can be manipulated such that the first width is reduced to a second width, wherein the second width is smaller than the second diameter of the internal passageway.

8. A system as in claim 7, wherein manipulation of the first width to the second width comprises rolling, folding, or bending of the elongate film.

9. A system as in claim 1, wherein the elongate film has a smooth finish.

10. A system as in claim 1, wherein the elongate film comprises a polymeric material or an elastic material.

11. A system as in claim 1, wherein the elongate film removably attaches to the spool core at a distal end of the elongate film.

12. A system as in claim 1, wherein the elongate film removably attaches to the spool core via at least one intermediary element.

13. A system as in claim 12, wherein the at least one intermediary element comprises a tether.

14. A system as in claim 1, wherein the spool core comprises at least one perimeter edge having an alignment structure that assists in alignment of the elongate film wound around the spool core.

15. A system as in claim 14, wherein the alignment structure comprises a raised flange.

16. A system as in claim 1, wherein the elongate film comprises at least one perimeter edge having an alignment structure that assists in alignment of the elongate film wound around the spool core.

17. A system as in claim 1, further comprising a locking member that engages the spool core and the elongate film wound around the spool core to lock the elongate film in a wound state around the spool core.

18. A system as in claim 17, wherein the elongate film has a series of holes that align with one another as the elongate film is wound around the spool core and wherein the holes collectively form a port that receives the locking member.

19. A method for distracting adjacent vertebral bodies, comprising:
introducing percutaneously a rotatable spool core into a portion of a disc space between the adjacent vertebral bodies;
introducing percutaneously an elongate film into the portion of the disc space between the adjacent vertebral bodies;
attaching the elongate film to the spool core to form a spooled assembly having a first diameter;
rotating the spool core to wind the film around the spool core;
increasing the first diameter of the spooled assembly to a second diameter by adding successive layers of the elongate film to the spool core, wherein the second diameter is greater than the first diameter; and
exerting a force by the spooled assembly against at least one of the adjacent vertebral bodies causing relative distraction of the adjacent vertebral bodies.

20. A method as in claim 19, wherein introducing percutaneously the rotatable spool core into the portion of the disc space comprises forming a first access channel on one side of a mid-sagittal plane of the disc and introducing the rotatable spool core into the disc space through the first access channel.

21. A method as in claim 19, wherein introducing percutaneously the elongate film into the portion of the disc space comprises forming a second access channel into the disc space on an opposite side of the mid-sagittal plane of the disc and introducing the elongate film into the disc space through the second access channel.

22. A method as in claim 20, wherein the first access channel comprises a first intraosseous transpedicular passageway.

23. A method as in claim 21, wherein the second access channel comprises a second intraosseous transpedicular passageway.

24. A method as in claim 19, further comprising locking the successive layers of film wound around the spool core.

25. A method as in claim 19, further comprising attaching percutaneously a drive shaft to the spool core, wherein the drive shaft introduces the spool core into the disc space.

26. A method as in claim 25, wherein the drive shaft rotates the spool core with an amount of torque.

27. A method as in claim 26, further comprising measuring the amount of torque used to rotate the spool core.

28. A method as in claim 27, wherein the amount of torque measured determines the desired extent of distraction between adjacent vertebral bodies.

29. A method as in claim 25, further comprising detaching the drive shaft from the spool core and removing the drive shaft from the disc space.

30. A sterile packaging system in which the elongate film of claim 1 is prepackaged in a wound sterile form.

31. A sterile packaging system as in claim 30, wherein the elongate film can be delivered percutaneously through an internal passageway of a film delivery device member inserted in an access channel.

32. A sterile packaging system as in clam 31, wherein a length of film delivered or retained by the packaging system can be determined by a visible indicator on the film.

33. A sterile packaging system as in claim 31, wherein a length of film delivered or retained by the packaging system can be determined by a visible indicator on or within a packaging enclosure.
